(19) United States
(12) Patent Application Publication
(10) Pub. No.: US 2026/0137392 A1
(43) Pub. Date: May 21, 2026

(54) SURGICAL CLIP AND APPLICATOR FOR SURGICAL CLIP

(71) Applicants: Pusan National University Industry-University Cooperation Foundation, Busan (KR); Pusan National University Hospital, Busan (KR)

(72) Inventors: Sangsu LEE, Yangsan-si (KR); Junyong JEKAL, Yangsan-si (KR)

(73) Assignees: Pusan National University Industry-University Cooperation Foundation, Busan (KR); Pusan National University Hospital, Busan (KR)

(21) Appl. No.: 19/391,386

(22) Filed: Nov. 17, 2025

(30) Foreign Application Priority Data
Nov. 19, 2024 (KR) 10-2024-0165283
Nov. 19, 2024 (KR) 10-2024-0165325

(51) Int. Cl. A61B 17/128; A61B 17/00; A61B 17/122

(52) U.S. Cl. CPC A61B 17/1285; A61B 17/122; A61B 2017/00862

(57) ABSTRACT

A surgical clip in which a fork body portion branched in the form of an inverted ‘U’ is formed at a rear portion and a single body portion curvedly extending forward from an upper middle portion of the fork body portion is formed at a front portion. The surgical clip has a generally C-shaped configuration with an open lower end such that the open lower end elastically opens and closes to elastically suture an incised tissue.

### CROSS-REFERENCE TO RELATED PATENT APPLICATION

This Application claims priority to Korean Patent Application Nos. 10-2024-0165283 (filed on Nov. 19, 2024) and 10-2024-0165325 (filed on Nov. 19, 2024), which are all hereby incorporated by reference in their entirety.

### BACKGROUND

The present disclosure relates to a surgical clip that may easily suture an incised intestinal tissue without invasive damage when the clip is pulled out from a side of a shaft in a laparoscopic surgery or intestinal anastomosis surgery situation and an applicator for using the surgical clip.

Preventing bleeding during surgical procedures is important not only for securing a surgical field but also for a patient's prognosis. Therefore, effective prevention of bleeding and hemostasis at an incision site during surgery are fundamentally necessary.

There are methods of ligation used, an electric coagulation method, a suture regulation method using a suture, and a method using a clip. Although the electric coagulation method is simple and easy to operate, it has a disadvantage of damaging surrounding a tissue due to the electric coagulation, and suturing takes a relatively long time. On the other hand, the method using the clip is widely used because it is simple to operate and may be removed at the end of the surgery, so there is no tissue damage and it helps with wound healing.

A conventionally used clip is made of an elastic material and is composed of a pair of legs that are connected at one end and open at the other end, and is used by opening one side of the legs and inserting a ligation site between the legs.

An example of a surgical clip that may suture an incised intestinal tissue during a laparoscopic surgery or an intestinal anastomosis surgery is a bio-absorbable clip in the form of a ‘C’ shape disclosed in U.S. Patent Application Publication No. US 2015/0080914 A1. The bio-absorbable clips are configured to be able to suture an incised intestinal tissue site by being pulled one by one out from the lowest portion of an applicator while a plurality of clips are mounted on the applicator.

However, conventional surgical clips such as those mentioned above inevitably cause invasive damage to a barrier, and as a result, there is a problem that they may damage the barrier by causing an inflammatory reaction and the like.

In addition, the conventional surgical clips suture a tissue by vertically positioning a shaft of the applicator containing the plurality of clips to a suture site and pulling the clips out in an axial direction through on an outlet at a lower end of the shaft, and this surgical method has a problem in that a tissue site that should be sutured may not be accurately caught by the clip and may come off during the process of the clip elastically opening and closing, making a suture work difficult.

### SUMMARY

The present disclosure is intended to solve the above-described problem, and provides a surgical clip having a structure that may securely suture an intestinal tissue while preventing invasive damage to a barrier.

Another object of the present disclosure is to provide a surgical clip applicator that enables more convenient intestinal anastomosis by allowing an incised intestine to be sutured while the clip is pulled out from a side of a shaft in a surgical situation.

According to one aspect of the present disclosure for achieving the above-described object, a surgical clip is characterized in that a fork body portion branched in the form of an inverted ‘U’ is formed at a rear portion and a single body portion curvedly extending forward from an upper middle portion of the fork body portion is formed at a front portion, and that the surgical clip has a generally C-shaped configuration with an open lower end such that the open lower end elastically opens and closes to elastically suture an incised tissue.

Inner surfaces of both branched sides of the fork body portion may be supported by a clip support portion protruding rearwardly from a front end of a clip fixing bar configured to move forward and backward in a surgical clip applicator, and a space between the both sides of the fork body portion may be a passage through which a front end of a clip expansion bar configured to move forward and backward in the surgical clip applicator passes to press the single body portion forward.

A bar engaging groove in which a tip end of the clip expansion bar is caught may be formed concavely on the inner surface of the single body portion of the clip.

Circular-rod-shaped fixing portions extending in a width direction of the surgical clip may be formed at lower ends of the fork body portion and the single body portion, so that the fixing portions press and suture the incised tissue.

According to another aspect of the present disclosure, there is provided a surgical clip applicator that elastically sutures an incised tissue portion using a plurality of clips, each having the above-described configuration, including a guide shaft in which a clip storage chamber, in which the plurality of clips are arranged in a row, is formed along a longitudinal direction, a clip inlet which the clip is inserted to a rear portion of the clip storage chamber is formed to be open, and a clip outlet which the clip is pulled out to one side of a front portion of the clip storage chamber is formed to be open laterally, a clip fixing bar installed to move in the longitudinal direction within the clip storage chamber and configured to support the clip at one side of the clip outlet, release a clip's fixed state while moving forward, and fix the clip while moving backward, and a clip expansion bar installed to move along the longitudinal direction within the clip storage chamber and configured to push a front portion of the clip forward to cause a lower end of the clip to open.

According to the present disclosure, circular-rod-shaped fixing portions are formed at the lower ends of a fork body portion and a single body portion, which together define a generally C-shaped configuration open downward. Since the tissue is sutured by the fixing portions, no invasive damage occurs during the suture process, and as a result, the advantage of preventing barrier damage caused by inflammatory reactions or the like may be obtained.

In addition, the surgical clip may include the fork body portion that is bifurcated into two branches and a single body portion. Through a space between the two branches of the fork body portion, a clip expansion bar of the applicator may advance to open the surgical clip. Accordingly, it becomes possible to withdraw the surgical clip laterally from the applicator, thereby achieving the effect of enabling the tissue to be sutured securely and easily.

In other words, after the surgical clip opens and closes inside a clip outlet of the applicator to completely suture the incised tissue, the fixed state of the clip is released, and the clip is discharged from the clip outlet. Accordingly, the effect of enabling the incised tissue to be sutured more securely and easily may be achieved.

In addition, the applicator for using the surgical clip of the present disclosure has a structure in which the clip outlet is formed on a side of a front end of a guide shaft so that the clip is pulled out through the side of the guide shaft to suture the tissue, thereby providing an advantage of allowing tissue suture to be performed reliably and easily.

In particular, since the clip is released from the fixed state and pulled out from the clip outlet after the incision tissue is completely sutured by opening and closing inside the clip outlet, there is an advantage in that the incision tissue may be sutured more reliably and easily.

### BRIEF DESCRIPTION OF THE DRAWINGS

FIG. 1 is a perspective view of a surgical clip according to one embodiment of the present disclosure.

FIG. 2 is a front view of the surgical clip illustrated in FIG. 1.

FIG. 3 is a perspective view of the surgical clip according to another embodiment of the present disclosure.

FIG. 4 is a front view showing a surgical clip applicator according to one embodiment of the present disclosure.

FIG. 5 is an enlarged front view showing a main portion of the surgical clip applicator illustrated in FIG. 4.

FIG. 6A and FIG. 6B are enlarged perspective views of portion A of FIG. 5.

FIG. 7 is a right side view of a clip fixing bar included in the surgical clip applicator illustrated in FIG. 4.

FIGS. 8A to 8D are drawings sequentially explaining an example of operation of the surgical clip applicator illustrated in FIG. 4.

FIG. 9 is a perspective view showing a partially cutaway portion of a surgical clip according to another embodiment of the present disclosure.

FIG. 10 is a cross-sectional view of a portion of the surgical clip illustrated in FIG. 9.

FIG. 11 is a cross-sectional view of another portion of the surgical clip illustrated in FIG. 9.

FIG. 12A and FIG. 12B are cross-sectional views showing an example of operation of the surgical clip illustrated in FIG. 9.

### DETAILED DESCRIPTION

Hereinafter, with reference to the accompanying drawings, a surgical clip and a surgical clip applicator according to preferred embodiments of the present disclosure will be described in detail.

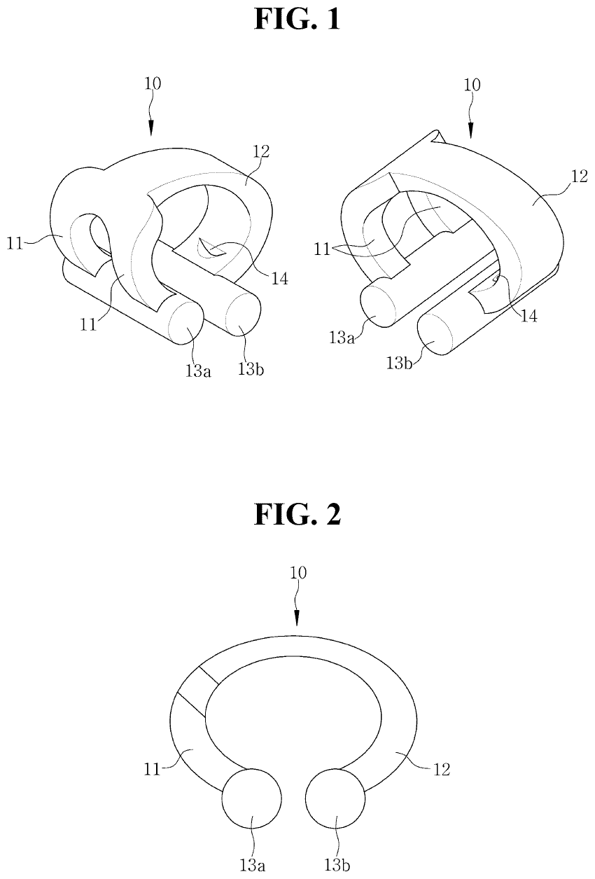

Referring to FIG. 1 and FIG. 2, a surgical clip 10 according to an embodiment of the present disclosure is suitable for use in suturing an incised intestinal tissue after surgery such as a laparoscopic surgery or an intestinal anastomosis surgery, and configured to be suitable for use in a surgical clip applicator, and has a generally C-shaped configuration with an open lower end and is configured to elastically suture an incised tissue portion through the open lower end.

For example, the surgical clip 10 has a fork body portion 11 bifurcated in an inverted ‘U’-shape is formed at a rear portion, a single body portion 12 curvedly extending forward from an upper middle portion of the fork body portion 11 formed at a front portion, and has a generally C-shaped configuration with the open lower end, so that the open lower end elastically opens and closes to elastically suture an incised tissue.

Circular-rod-shaped fixing portions 13a, 13b extending in a width direction of the clip are integrally formed at a lower end of the fork body portion 11 and a lower end of the single body portion 12 and the fixing portions 13a, 13b press and suture an incised site of a tissue. In this way, in the case that the circular-rod-shaped fixing portions 13a, 13b are formed at the lower end of the fork body portion 11 and the lower end of the single body portion 12, invasive damage does not occur when the tissue is sutured by the fixing portions 13a, 13b, and as a result, an advantage of preventing damage to a barrier due to an inflammatory reaction, etc. may be obtained.

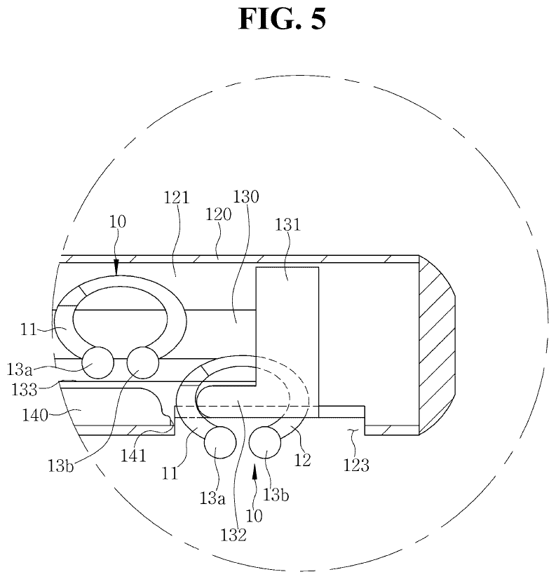

The fork body portion 11 is formed with a width greater than a width of the single body portion 12 so that both sides protrude outward in a width direction of the single body portion 12 and inner surfaces of the both sides of the fork body portion 11 are supported by being caught on a clip holder 131 (see FIG. 5) formed at a front end of a clip fixing bar 130 (see FIG. 5) formed in a surgical applicator. In addition, since the both sides of the fork body portion 11 are separated from each other to form a space, a front end of a clip expansion bar 140 (see FIG. 5) configured in the surgical applicator may pass through the space between the both sides of the fork body portion 11 and press an inner surface of the single body portion 12 forward.

A bar engaging groove 14 is concavely formed on the inner surface of the single body portion 12 of the surgical clip 10 in which a tip end of the clip expansion bar 140 is caught (see FIG. 5) so that when the clip expansion bar 140 moves forward to push the single body portion 12 forward, the single body portion 12 is smoothly pushed forward without slipping, so that the lower end of the surgical clip 10 may be opened.

The surgical clip 10 is made of a bioabsorbable material that may naturally dissolve and disappear over time in a human body, and thus does not cause foreign body reactions or adhesions in the human body. It is also designed to have enough an elastic force to secure the barrier without affecting the blood flow to the intestine.

According to the surgical clip 10 having such a configuration, the inner surface of the rear fork body portion 11 is supported by the clip fixing bar 130 (see FIG. 5) of the surgical applicator, and the single body portion 12 passes between the both sides of the fork body portion 11 and is pressed forward by the front end of the clip expansion bar 140 (see FIG. 5) of the surgical applicator, thereby causing the lower ends of the fork body portion 11 and the single body portion 12 to open.

At this time, after placing a gap of the open lower end of the fork body portion 11 and the single body portion 12 on the tissue to be sutured, and the clip expansion bar 140 (see FIG. 5) is moved backward to remove an external force applied to the single body portion 12 the single body portion 12 is returned to its original state by the elastic force, and the fixing portions 13a, 13b of the lower end firmly suture the tissue.

Conventional suturing methods that suture the barrier of a large intestine or a small intestine with needles or metal pins, or conventional clips (e.g., the clip disclosed in U.S. Patent Application Publication No. US 2015/0080914 A1) inevitably cause invasive damage to the barrier, which may result in damage to the barrier due to an inflammatory reactions, etc., but the surgical clip 10 of the present disclosure sutures the tissue with the circular-rod-shaped fixing portions 13a, 13b at the lower ends, so it has the advantage of preventing damage to the barrier due to the inflammatory reactions, etc. without causing invasive damage to the barrier.

In addition, since the surgical clip 10 has a structure suitable for use in the surgical clip applicator of the present disclosure, a plurality of surgical clips 10 may be used to easily suture the tissue using the surgical clip applicator of the present disclosure.

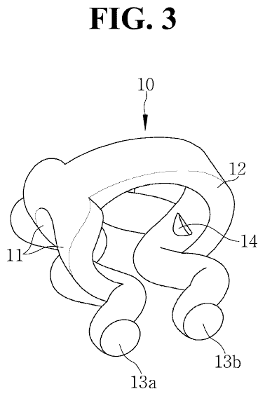

As in this embodiment, the fixing portions 13a, 13b may have a straight circular-rod shape, but as shown in another embodiment in FIG. 3, they may also be formed in a circular-rod shape having a zigzag or wave pattern so that they may be interlocked with each other when suturing the incision tissue.

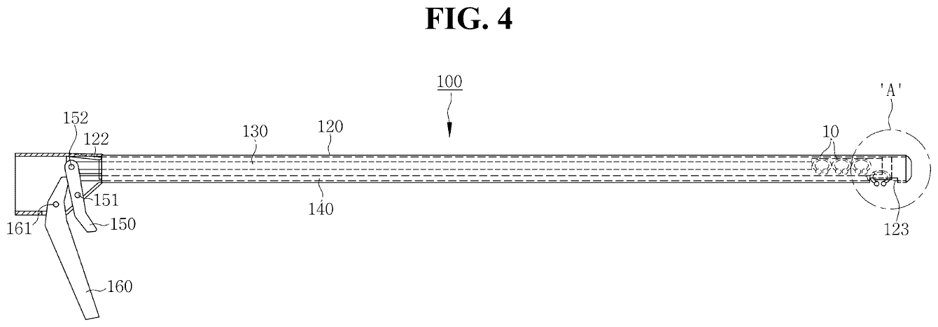

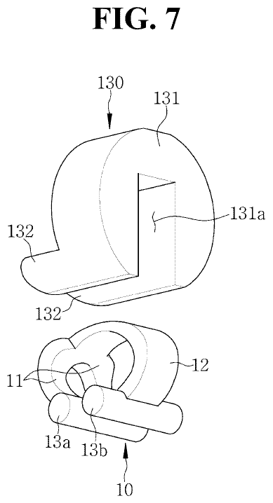

FIGS. 4 to 8 illustrate one embodiment of an applicator 100 having a structure suitable for using the above-described surgical clip 10.

The applicator 100 may include a main body portion 110, a guide shaft 120 formed to extend forward of the main body portion 110, a clip fixing bar 130 and a clip expansion bar 140 installed to move along a longitudinal direction within the guide shaft 120, a release operating member 150 installed in the main body portion 110 to move the clip fixing bar 130 forward and backward in response to a surgeon's operation, and a separation operating member 160 to move the clip expansion bar 140 forward and backward.

The main body portion 110 has a size and form that may be held and operated by a surgeon's hand, and supports the guide shaft 120 the release operating member 150 and the separation operating member 160. The guide shaft 120 is formed to extend forward at a front end of the main body portion 110.

The guide shaft 120 has an elongated circular or polygonal rod shape in which a clip storage chamber 121, in which the plurality of surgical clips 10 are arranged in a row, is formed along the longitudinal direction. A clip inlet 122 is formed openly at a rear portion of the guide shaft 120 and is connected to a rear portion of the clip storage chamber 121 and a clip outlet 123 which the surgical clip 10 is pulled out is formed open laterally at one side of a front portion of the clip storage chamber 121.

The clip fixing bar 130 is installed in the form of a long bar shape in a front-back direction to move along the longitudinal direction within the clip storage chamber 121 of the guide shaft 120. A rear end of the clip fixing bar 130 is connected to the release operating member 150 within the main body portion 110 and moves linearly back and forth in the front-back direction by the release operating member 150. The clip holder 131 is formed at the front end of the clip fixing bar 130 to support the surgical clip 10 located at the frontmost position among the surgical clips 10 just inside the clip outlet 123. Accordingly, the clip holder 131 separates from the surgical clip 10 so that the clip fixing bar 130 moves forward and, the surgical clip 10 is pulled out to a outside of the clip outlet 123. And when the clip fixing bar 130 moves backward, the clip holder 131 supports and fixes the surgical clip 10 that has moved inside the clip outlet 123.

A guide groove 133 may be formed concavely along the longitudinal direction at a lower end of one side of the clip fixing bar 130 to guide one portion of the fixing portions 13a, 13b of the lower end of the surgical clip 10 as it is inserted.

The clip holder 131 includes a clip receiving portion 131a formed in the form of an inverted ‘U’ at the front end of the clip fixing bar 130 to receive the single body portion 12 of the clip, and a clip support portion 132 that extends rearward by a predetermined distance from a lower end of the clip receiving portion 131a and supports the surgical clip 10 by contacting the inner surfaces of the both sides of the fork body portion 11. An end of the clip support portion 132 is curved and is in close contact with the inner surfaces of the both sides of the fork body portion 11 of the surgical clip 10 thereby fixing the surgical clip 10 inside the clip outlet 123.

The clip fixing bar 130 is configured to move along the longitudinal direction (front-back direction) of the guide shaft 120 by a force of operation applied by a user to the release operating member 150 provided in the main body portion 110. For example, the clip fixing bar 130 is in the form of a trigger or lever and is connected to an upper portion of the release operating member 150 installed to rotate around a first hinge axis 151 at a front portion of the main body portion 110 through a connecting pin 152 so that the release operating member 150 moves along the longitudinal direction of the guide shaft 120 as it rotates around the first hinge axis 151. The release operating member 150 or the clip fixing bar 130 is elastically supported by a spring not shown in the drawing, so that when the user removes a force pressing the release operating member 150, the clip fixing bar 130 moves backwards by the elastic force of the spring and returns to its original position.

The clip expansion bar 140 is installed to move in the longitudinal direction (front-back direction) within the clip storage chamber 121 and acts to push the single body portion 12 formed at the front of the surgical clip 10 forward to cause the lower end of the surgical clip 10 to open. A clip engaging protrusion 141 that is inserted into and caught in the bar engaging groove 14 formed on the inner surface of the single body portion 12 of the surgical clip 10 may be formed to protrude forward at a lower end of the tip end of the clip expansion bar 140.

The clip expansion bar 140 is in the form of a trigger or lever and is moved forward along the longitudinal direction of the guide shaft 120 by the separation operating member 160 installed in the main body portion 110 to rotate around a second hinge axis 161. The separation operating member 160 may be placed at a rear of the release operating member 150 in the main body portion 110 and an upper portion of the separation operating member 160 may be in contact with or connected with a pin or the like with a rear end of the clip expansion bar 140. Therefore, when a surgeon presses the separation operating member 160 the upper portion of the separation operating member 160 presses the rear end of the clip expansion bar 140 causing the clip expansion bar 140 to move forward. The separation operating member 160 or the clip expansion bar 140 is elastically supported by the spring not shown in the drawing, so that when the user removes the force pressing the separation operating member 160 the clip expansion bar 140 moves backwards by the elastic force of the spring and returns to its original position.

Although not shown in the drawing, the applicator 100 of the present disclosure may further include a clip transfer module that transfers the plurality of surgical clips 10 aligned in a row inside the guide shaft 120 forward by a predetermined distance. The clip transfer module applies pressure forward to the surgical clip 10 arranged at the rearmost position among the plurality of surgical clips 10 aligned in a row inside the clip storage chamber 121 of the guide shaft 120 thereby causing the surgical clip 10 at the frontmost position to be pulled out to an outside through the clip outlet 123 and then causes a next surgical clip 10 to move inside the clip outlet 123.

An applicator 100 having this configuration may operate as follows.

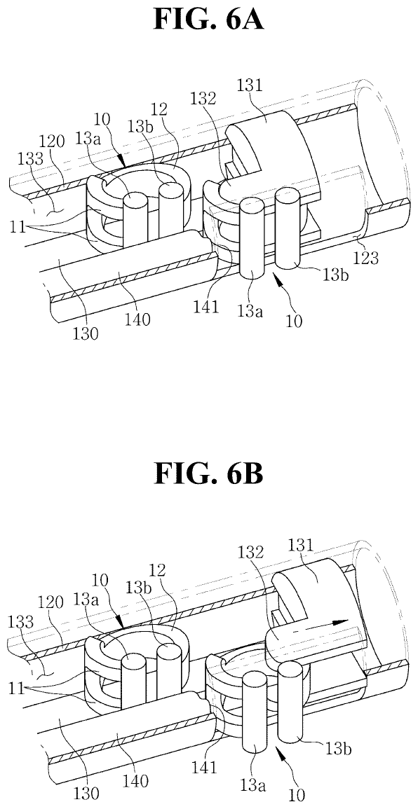

When the plurality of surgical clips 10 are inserted one by one through the clip inlet 122 formed at the rear end of the clip storage chamber 121 of the guide shaft 120 the plurality of surgical clips 10 are arranged in a row within the clip storage chamber 121. At this time, the inner surface of the fork body portion 11 of the frontmost surgical clip 10 is supported by the clip holder 131 just inside the clip outlet 123 and in this state, two sets of the fixing portions 13a, 13b formed at a lower end of the frontmost surgical clip 10 protrude to the outside of the clip outlet 123 (see FIG. 6).

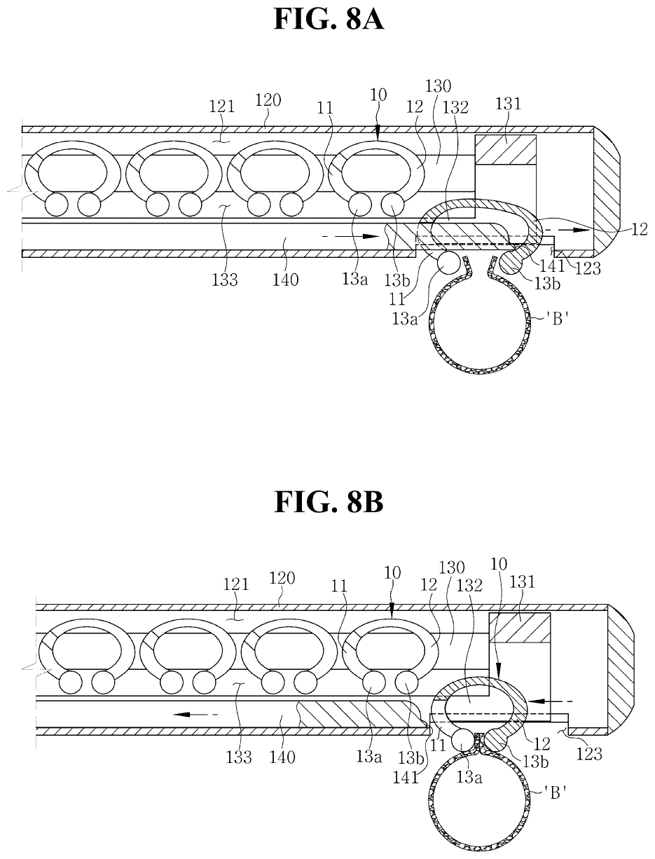

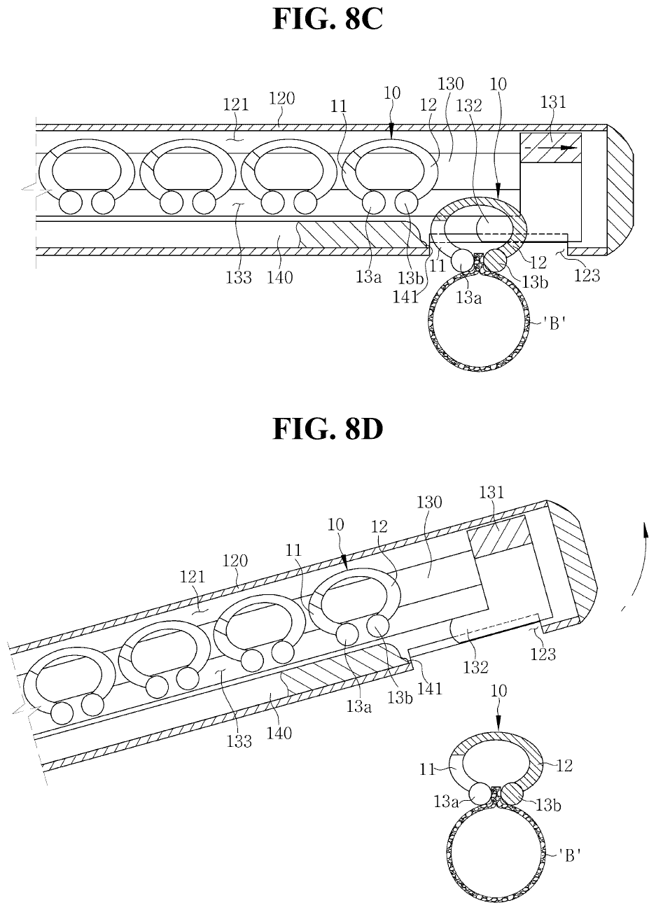

The surgeon inserts the guide shaft 120 into the human body, aligns the fixing portions 13a, 13b of the surgical clip 10 protruding outside the clip outlet 123 over the incision tissue to be sutured, and then when presses the separation operating member 160 the clip expansion bar 140 moves forward. As shown in FIG. 8A, when the clip expansion bar 140 moves forward, the front end of the clip expansion bar 140 passes between the both sides of the fork body portion 11 of the frontmost surgical clip 10 and contacts the inner surface of the single body portion 12 thereby pressing the single body portion 12 forward. At this time, the clip engaging protrusion 141 at the tip of the clip expansion bar 140 is caught as it is inserted into the inner surface of the single body portion 12.

In this state, when the clip expansion bar 140 moves forward a predetermined distance, the single body portion 12 and the fixing portion 13b at the lower end of the single body portion 12 are pushed forward, and the fixing portions 13a, 13b at the lower end of the surgical clip 10 are open, and when the tissue to be sutured is inserted between the open two fixing portions 13a, 13b and the force pressing the separation operating member 160 is removed, the clip expansion bar 140 moves backward and returns to the original position by the elastic force of the spring (not shown) as shown in FIG. 8B. Accordingly, the force pressing the single body portion 12 of the surgical clip 10 is released, the fixing portions 13a, 13b are closed, and the incised tissue between the two fixing portions 13a, 13b is bitten and sutured. At this time, as described above, the fixing portions 13a, 13b are formed in a circular-rod shape, so the incised tissue is not subjected to invasive damage.

When the surgical clip 10 opens and closes and the tissue is sutured by the fixing portions 13a, 13b, the surgeon presses the release operating member 150. When the release operating member 150 is pressed, the clip fixing bar 130 moves forward, and as shown in FIG. 8C, a rear end of the clip holder 131 and the inner surfaces of the fork body portion 11 of the surgical clip 10 are separated from each other, and the surgical clip 10 becomes free. At this time, when the surgeon lifts the guide shaft 120, the surgical clip 10 is pulled out from the clip outlet 123 as shown in FIG. 8D.

And when the surgeon removes the force pressing the release operating member 150, the clip fixing bar 130 moves backwards by the elastic force of the spring (not shown) and returns to its original position, moves inside the clip outlet 123 and then supports the inner surfaces of the both sides of the fork body portion 11 of the surgical clip 10.

Then, when the surgical clip 10 slides along the inside of the clip storage chamber 121 and moves toward the clip outlet 123, the inner surfaces of the both sides of the fork body portion 11 of the surgical clip 10 is supported by contacting the rear end of the clip holder 131 as described above, and is fixed inside the clip outlet 123.

When the clip transfer module is not configured in the clip applicator 100, the plurality of surgical clips 10 arranged inside the clip storage chamber 121 naturally slide forward by their own weight by an action of tilting the front end of the guide shaft 120 downward, so that the frontmost surgical clip 10 moves inside the clip outlet 123 and is fixed by the clip holder 131 and a suturing operation using the next surgical clip 10 may be prepared.

The surgeon may use the plurality of surgical clips 10 to suture the incised tissue by continuously repeating these operations.

In this way, the surgical clip applicator has the clip outlet 123 formed on a side of the front end of the guide shaft 120 and the surgical clip 10 is pulled out through the side of the guide shaft 120 to suture the tissue, thereby providing the advantage of being able to perform the tissue suturing reliably and easily.

In addition, the surgical clip 10 opens and closes inside the clip outlet 123 to completely suture the tissue, and then is released from the fixed state and pulled out from the clip outlet 123 so that the suture of the incision tissue may be performed more reliably and easily.

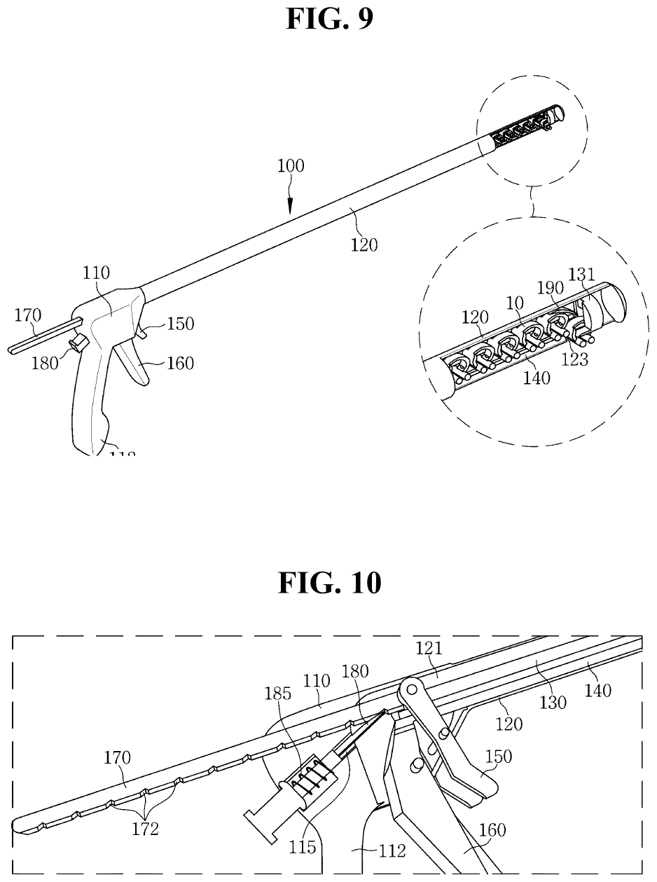

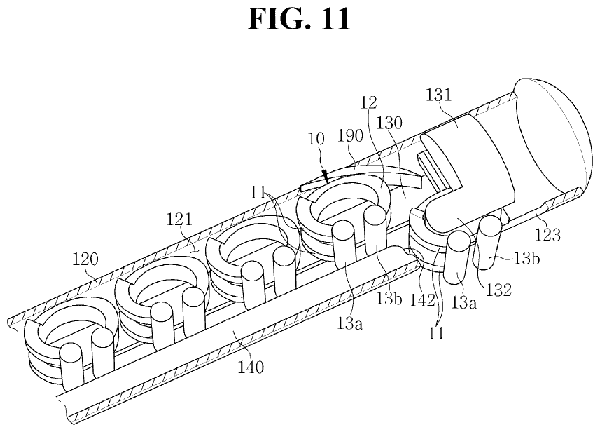

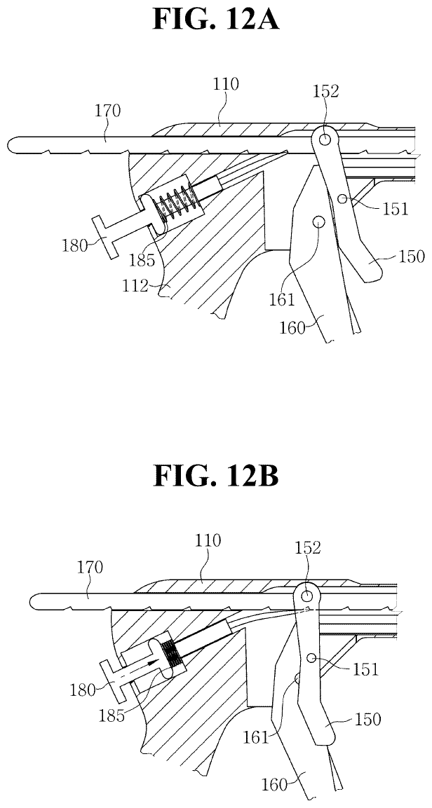

FIGS. 9 to 12 illustrate a second embodiment of the surgical clip applicator 100 according to the present disclosure, the surgical clip applicator 100 of this second embodiment is different from the surgical clip applicator 100 of the first embodiment described above in that a handle 112 is formed to extend downwardly so that the user may hold it by hand in the main body portion 110, a clip push module is installed in the main body portion 110 to push forward the plurality of clips 10 stored in the guide shaft 120 one by one, and a clip guide 190 is installed in a front end of the clip storage chamber 121 of the guide shaft 120 to guide the clips 10 to the clip outlet 123.

Inside the clip storage chamber 121 of the guide shaft 120, the clip fixing bar 130 and a clip expansion bar 140 are installed to fix and release the frontmost clip 10 located inside the clip outlet 123 within the clip outlet 123 and to forcibly open the clip 10. Further, the release operation member 150 and the separation operating member 160 that move the clip fixing bar 130 and the clip expansion bar 140 forward by the surgeon's operation are installed in the main body portion 110.

The configuration and operation of the clip fixing bar 130 and the clip expansion bar 140, the release operating member 150 and the separation operating member 160 are the same as the configuration and operation of the clip fixing bar 130 and the clip expansion bar 140, the release operating member 150 and the separation operating member 160 configured in the surgical clip applicator 100 of the first embodiment described above, so a detailed description thereof is omitted.

The clip push module is configured to push the plurality of clips 10 aligned in a row inside the clip storage chamber 121 of the guide shaft 120 forward at a predetermined pitch, so that, after the frontmost clip 10 located in the clip outlet 123 is pulled out through the clip outlet 123, the next clip 10 is moved to the clip outlet 123.

For example, the clip push module may include a clip push bar 170 that is installed so as to be slidable in the longitudinal direction (front-back direction) inside the clip storage chamber 121 of the guide shaft 120 and is in contact with the rearmost clip 10 of the plurality of clips 10, a push operation pin 180 that is installed to move obliquely forward and backward along a pin guide passage 115 inclined at a predetermined angle on the main body portion 110, a tip end of the push operation pin 180 being inserted into a push groove 172 formed at predetermined intervals on a lower surface of the clip push bar 170, the push operation pin 180 being configured to move the clip push bar 170 forward while moving obliquely forward and backward at a predetermined angle in response to a pressing operation by the surgeon, and a spring 185 that elastically supports the push operation pin 180 inside the pin guide passage 115 of the main body portion 110.

The push operation pin 180 is configured to be able to bend or move to a certain degree while moving forward, so that the push operation pin 180 may move the clip push bar 170 forward moving obliquely forward.

Accordingly, immediately after the frontmost clip 10 sutures the incision tissue and is pulled out through the clip outlet 123 by the operation of the clip expansion bar 140 and the clip fixing bar 130, when the surgeon presses a rear end of the push operation pin 180 protruding from the rear end of the main body portion 110 the clip push bar 170 moves forward in an upwardly inclined direction at a predetermined angle, thereby pushing the clip push bar 170 forward, and as the clip push bar 170 moves forward a predetermined distance, it pushes the row of clips 10 forward a predetermined distance. Accordingly, the frontmost clip 10 moves inside the clip outlet 123 and is fixed by the clip holder 131 of the clip fixing bar 130.

When the surgeon removes the force that was pressing the push operation pin 180 returns to its original position due to the elastic force of the spring 185 and the tip end is inserted into the inside of another push groove 172 arranged on the lower surface of the clip push bar 170 thereby supporting the clip push bar 170 from being pushed backward.

Thus, in order to accurately transfer the frontmost clip 10 to a predetermined position inside the clip outlet 123 when the clip 10 is moved forward by the clip push module, the curved clip guide 190 is installed on a side opposite to the clip outlet 123.

The clip guide 190 guides the clip 10 toward the lower clip outlet 123, while contacting the front portion of the frontmost clip 10, that is the single body portion 12, that moves by the clip push module,

Although the detailed description of the present disclosure described above has been described with reference to preferred embodiments of the present disclosure, it will be understood by those skilled in the art or having ordinary knowledge in the art that various modifications and changes can be made to the present disclosure without departing from the spirit and technical scope of the present disclosure as set forth in the appended claims to be described later.

1. A surgical clip in which a fork body portion branched in the form of an inverted ‘U’ is formed at a rear portion and a single body portion curvedly extending forward from an upper middle portion of the fork body portion is formed at a front portion, wherein the surgical clip has a generally C-shaped configuration with an open lower end such that the open lower end elastically opens and closes to elastically suture an incised tissue.

2. The surgical clip according to claim 1, wherein inner surfaces of both branched sides of the fork body are supported by a clip support portion protruding rearwardly from a front end of a clip fixing bar configured to move forward and backward in a surgical clip applicator, and a space between the both sides of the fork body portion is a passage through which a front end of a clip expansion bar configured to move forward and backward in the surgical clip applicator passes to press the single body portion forward.

3. The surgical clip according to claim 2, wherein a bar engaging groove in which a tip end of the clip expansion bar is caught is concavely formed on an inner surface of the single body portion of the clip.

4. The surgical clip according to claim 1, wherein circular-rod-shaped fixing portions extending in a width direction of the surgical clip are formed at lower ends of the fork body portion and the single body portion, so that the fixing portions press and suture the incised tissue.

5. The surgical clip according to claim 4, wherein each of the fixing portions has a straight circular-rod shape.

6. The surgical clip according to claim 4, wherein each of the fixing portions has a circular-rod shape formed in a zigzag or wave pattern so as to engage with each other.

7. A surgical clip applicator for elastomeric suturing of an incised tissue portion using a plurality of clips having a configuration according to claim 1, the surgical clip applicator comprising:
a guide shaft in which a clip storage chamber, in which the plurality of clips are arranged in a row, is formed along a longitudinal direction, a clip inlet through which clips are inserted is formed to be open at a rear portion of the clip storage chamber, and a clip outlet through which clips are pulled out is formed to be open laterally at one side of a front portion of the clip storage chamber;
a clip fixing bar installed to move in the longitudinal direction within the clip storage chamber and configured to support the clip at one side of the clip outlet, release a clip's fixed state while moving forward, and fix the clip while moving backward; and
a clip expansion bar installed to move along the longitudinal direction within the clip storage chamber and configured to push a front portion of the clip forward to cause a lower end of the clip to open.

8. The surgical clip applicator according to claim 7, wherein a clip holder of the clip fixing bar comprises a clip receiving portion formed in an inverted ‘U’-shape at a front end of the clip fixing bar to receive the single body portion of the clip, and a clip supporting portion extending rearward from a lower end of the clip receiving portion to contact and support inner surfaces of both sides of the fork body portion of the clip.

9. The surgical clip applicator according to claim 7, wherein a bar engaging groove in which a tip end of the clip expansion bar is caught is concavely formed on an inner surface of the single body portion of the clip.

10. The surgical clip applicator according to claim 7, wherein a main body portion that is able to be held manually and operated by a surgeon is provided at a rear end of the guide shaft, and the main body portion is provided with a release operating member configured to move the clip fixing bar forward and backward in response to an operation of the surgeon and a separation operating member configured to move the clip expansion bar forward and backward.

11. The surgical clip applicator according to claim 10, wherein the release operating member and the separation operating member are each formed in the shape of a trigger or a lever rotatably installed in the main body portion, and are elastically supported by an elastic member so that the release operating member and the separation operating member automatically return to their original positions by an elastic force of the elastic member after they move forward.

12. The surgical clip applicator according to claim 10, further comprising a clip push module configured to move a plurality of clips arranged in a row in the clip storage chamber of the guide shaft forward a predetermined distance after the frontmost clip located in the clip outlet is pulled out through the clip outlet.

13. The surgical clip applicator according to claim 12, wherein the clip push module comprises:
a clip push bar that is installed so as to be slidable along the longitudinal direction inside the main body portion and the clip storage chamber, a front end of the clip push bar being in contact with a rearmost one of the plurality of clips;
a push operation pin that is installed to move obliquely forward and backward along a passage inclined at a predetermined angle on the main body portion, a tip end of the push operation pin being inserted into a push groove formed at predetermined intervals on a lower surface of the clip push bar, the push operation pin being configured to move the clip push bar forward while moving obliquely forward and backward at a predetermined angle in response to a pressing operation by a surgeon; and
a spring that elastically supports the push operation pin inside the passage of the main body portion.

14. The surgical clip applicator according to claim 7, further comprising a curved clip guide installed inside a front end of the clip storage chamber on a side opposite to the clip outlet to contact an upper portion of the clip and guide the clip toward the clip outlet.
